(19) United States
(12) Patent Application Publication
(10) Pub. No.: US 2016/0279002 A1
(43) Pub. Date: Sep. 29, 2016

(54) ABSORBENT ARTICLES HAVING NONWOVEN SUBSTRATES WITH REACTIVE INK COMPOSITIONS

(71) Applicant: The Procter & Gamble Company, Cincinnati, OH (US)

(72) Inventors: Linda Ann SAUER, Cincinnati, OH (US); Clarissa Maldonado, Cincinnati, OH (US); Kyle James Raabe, Springfield Township, OH (US)

(21) Appl. No.: 15/076,696

(22) Filed: Mar. 22, 2016

Related U.S. Application Data
(60) Provisional application No. 62/139,434, filed on Mar. 27, 2015.

(51) Int. Cl. A61F 13/84; A61L 15/22; A61L 15/28; A61L 15/24

(52) U.S. Cl. CPC A61F 13/84; A61L 15/24; A61L 15/225; A61L 15/28; A61F 2013/8497

(57) ABSTRACT

An absorbent article comprising a nonwoven substrate having an ink printed inner side and a non-printed garment facing side and a reactive ink composition printed on the nonwoven substrate on the ink printed inner side, forming an ink printed area, wherein the reactive ink composition comprises a binder.

### FIELD OF THE INVENTION

The present invention relates to the printing of nonwoven substrates with a reactive ink composition. Specifically, the present invention relates to printed nonwoven substrates that are printed with a reactive ink resulting in improved in-use properties.

### BACKGROUND OF THE INVENTION

The printing of substrates, such as woven and nonwoven fabrics and films, is well known. The printing of fabrics with inks and dyes is a common and widely used method for imparting patterns and colors to a basic fabric. Many current products, such as diapers and training pants, include printed designs to improve their appearance. A problem with such printed products is that the printed design can be smeared or even be removed during the handling of products during manufacturing, packaging, and use.

Nonwoven printing is generally difficult and has issues of poor print quality, low image vibrancy, poor color-to-color registration, poor print pitch control, and poor ink adhesion, especially in cases of high ink density (>0.3). Thus, there remains a need for a printed nonwoven substrate with improved print quality, ink density, and ink adhesion that may be used to manufacture a disposable absorbent article.

### SUMMARY OF THE INVENTION

The present invention relates to absorbent articles comprising a nonwoven substrate having an ink printed inner side and a non-printed garment facing side and a reactive ink composition printed on the nonwoven substrate on the ink printed inner side, forming an ink printed area, wherein the reactive ink composition comprises a binder and an organic solvent.

### BRIEF DESCRIPTION OF THE DRAWINGS

FIG. 1 is a plan view of a diaper in accordance with an embodiment of the present invention.

FIG. 2 is a cross sectional view of the diaper shown in FIG. 1 taken along the sectional line 2-2 of FIG. 1.

FIG. 3 is a partial cross sectional view of an absorbent core layer in accordance with an embodiment of this invention.

FIG. 4a is a partial sectional view of an absorbent core comprising a combination of the first and second absorbent core layers.

FIG. 4b is a partial sectional view of an absorbent core comprising a combination of the first and second absorbent core layers.

### DETAILED DESCRIPTION OF THE INVENTION

The present invention concerns the printing of nonwoven substrates with a reactive ink composition. Specifically, the substrates are useful in an absorbent article, such as a diaper.

As used herein, the following terms have the following meanings:

“Absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” means a structure typically disposed between a top sheet and cover sheet of an absorbent article for absorbing and containing liquid received by the absorbent article and may comprise one or more substrates, absorbent polymer material disposed on the one or more substrates, and a thermoplastic composition on the absorbent particulate polymer material and at least a portion of the one or more substrates for immobilizing the absorbent particulate polymer material on the one or more substrates. In a multilayer absorbent core, the absorbent core may also include a cover layer. The one or more substrates and the cover layer may comprise a nonwoven. Further, the absorbent core may be substantially cellulose free. The absorbent core does not include an acquisition system, a top sheet, or a back sheet of the absorbent article. In a certain embodiment, the absorbent core would consist essentially of the one or more substrates, the absorbent polymer material, the thermoplastic composition, and optionally the cover layer.

“Airfelt” is used herein to refer to comminuted wood pulp, which is a form of cellulosic fiber.

“Binder” refers to any substance in an ink that allows the ink pigment to adhere to the substrate, or printed surface, or to keep the pigment uniformly dispersed in the fluid ink vehicle.

“Colorant” includes one or more of pigments and or dyes; colorant may further include an acrylic colloidal dispersion, acrylic solution, or surfactants and water.

“Color Density” and “Optical Density,” otherwise referred to as “ink density”, is a function of the percentage of light reflected from a printed patch of printed ink. Ink Density is a unitless value correlating to the vibrancy of the ink printed onto a substrate. A low percentage of light reflected results in a high density. Data derived herein is based on an X-rite eXact Spectrophotometer for measurements.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Crosslinking agent”, “Crosslinking additive”, or “Curing agent” refers to a chemical deposited into the ink before or during printing which is capable of reacting with the binder to form covalent bonds thereby linking two or more binder molecules.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Fiber” and “filament” are used interchangeably.

“Ink” and “composition” are used interchangeably herein; an ink or composition may or may not include a colorant.

“Nitrocellulose” refers to cellulose which has any degree of nitration.

A “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwovens may include hydroentangled nonwovens. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999.

“Poly-epoxy compound” refers to a chemical compound with two or more epoxy groups.

“Poly-isocyanate” refers to a chemical compound comprising two or more isocyanate groups.

“Reactive ink”, otherwise referred to as curable ink or crosslinkable ink, refers to an ink containing a binder which is capable of being crosslinked when combined with a crosslinking agent.

“Substantially cellulose free” is used herein to describe an article, such as an absorbent core, that contains less than 10% by weight cellulosic fibers, less than 5% cellulosic fibers, less than 1% cellulosic fibers, no cellulosic fibers, or no more than an immaterial amount of cellulosic fibers. An immaterial amount of cellulosic material would not materially affect the thinness, flexibility, or absorbency of an absorbent core.

“Substantially continuously distributed”, as used herein, refers to absorbent particulate polymer material that is arranged across the absorbent particulate polymer material area. Optionally, the absorbent particulate polymer material may be arranged such that the substrate layers do not touch in various zones. In one embodiment, the substrate layers may touch in the peripheral areas outside the absorbent particulate polymer material area. It is important to note that the thermoplastic material used in the present invention does not interrupt the substantially continuously distributed absorbent particulate polymer material. Thus, the substantially continuously distributed absorbent particulate polymer material includes the thermoplastic material.

“Substrate” includes any material that the inks of the present invention can be printed on. Thus, substrates of the present invention include, but are not limited to, non-wovens, films, fibrous polyolefin webs, polyolefin webs, cellulosic webs, elastomeric webs, laminates of one or more of the above or any combination of one or more of the above.

“Saline Solution” refers to a solution of 0.9% Sodium Chloride prepared with 9.000 grams (+0.1 gram) of Sodium Chloride in 1 liter (+0.001 liter) of deionized water.

“Thickness” and “caliper” are used herein interchangeably.

“Wt %” refers to the percentage weight of a specific component relative to the entire composition.

### Absorbent Article

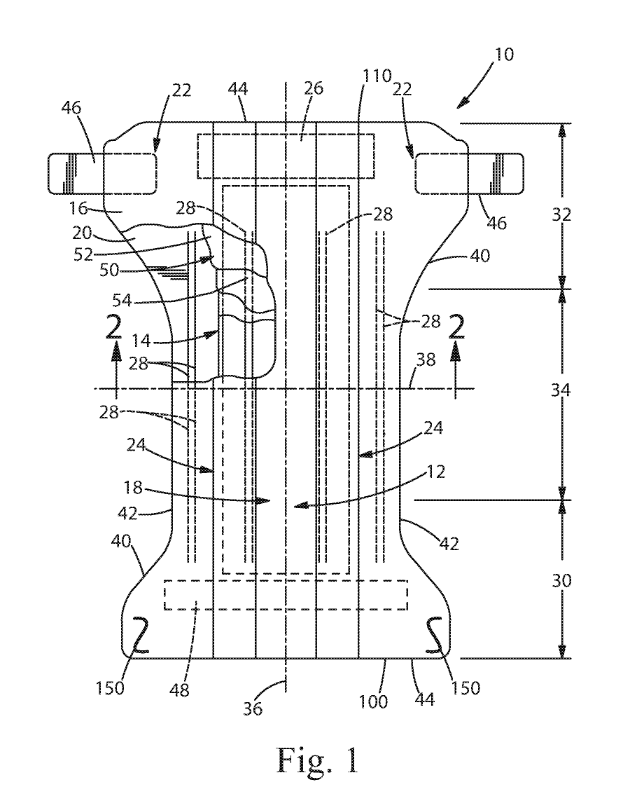

FIG. 1 is a plain view of a diaper 10 according to a certain embodiment of the present invention. The diaper 10 is shown in its flat out, uncontracted state (i.e., without elastic induced contraction) and portions of the diaper 10 are cut away to more clearly show the underlying structure of the diaper 10. A portion of the diaper 10 that contacts a wearer is facing the viewer in FIG. 1. The diaper 10 generally may comprise a chassis 12 and an absorbent core 14 disposed in the chassis.

The chassis 12 of the diaper 10 in FIG. 1 may comprise the main body of the diaper 10. The chassis 12 may comprise an outer covering 16 including a top sheet 18, which may be liquid pervious, and/or a back sheet 20, which may be liquid impervious. The absorbent core 14 may be encased between the top sheet 18 and the back sheet 20. The chassis 12 may also include side panels 22, elasticized leg cuffs 24, and an elastic waist feature 26.

The leg cuffs 24 and the elastic waist feature 26 may each typically comprise elastic members 28. One end portion of the diaper 10 may be configured as a first waist of the diaper 10. The first waist region 30 has a first edge 100. An opposite end portion of the diaper 10 may be configured as a second waist region 32 of the diaper 10. The second waist region 32 has a second edge 110. An intermediate portion of the diaper 10 may be configured as a crotch region 34, which extends longitudinally between the first and second waist regions 30 and 32. The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 is depicted in FIG. 1 with its longitudinal axis 36 and its transverse axis 38. The periphery 40 of the diaper 10 is defined by the outer edges of the diaper 10 in which the longitudinal edges 42 run generally parallel to the longitudinal axis 36 of the diaper 10 and the end edges 44 run between the longitudinal edges 42 generally parallel to the transverse axis 38 of the diaper 10. The chassis 12 may also comprise a fastening system, which may include at least one fastening member 46 and at least one stored landing zone 48.

The diaper 20 may also include such other features as are known in the art including front and rear ear panels, waist cap features, elastics and the like to provide better fit, containment and aesthetic characteristics. Such additional features are well known in the art and are e.g., described in U.S. Pat. No. 3,860,003 and U.S. Pat. No. 5,151,092.

In order to keep the diaper 10 in place about the wearer, at least a portion of the first waist region 30 may be attached by the fastening member 46 to at least a portion of the second waist region 32 to form leg opening(s) and an article waist. When fastened, the fastening system carries a tensile load around the article waist. The fastening system may allow an article user to hold one element of the fastening system, such as the fastening member 46, and connect the first waist region 30 to the second waist region 32 in at least two places. This may be achieved through manipulation of bond strengths between the fastening device elements.

According to certain embodiments, the diaper 10 may be provided with a re-closable fastening system or may alternatively provided in the form of a pant-type diaper. When the absorbent article is a diaper, it may comprise a re-closable fastening system joined to the chassis for securing the diaper to a wearer. When the absorbent article is a pant-type diaper, the article may comprise at least two side panels joined to the chassis and to each other to form a pant. The fastening system and any component thereof may include any material suitable for such a use, including but not limited to plastics, films, foams, nonwoven webs, woven webs, paper, laminates, fiber reinforced plastics and the like, or combinations thereof. In certain embodiments, the materials making up the fastening device may be flexible. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the diaper 10 with other features added to form the composite diaper structure. While the top sheet 18, the back sheet 20, and the absorbent core 14 may be assembled in a variety of well-known configurations, diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

The top sheet 18 in FIG. 1 may be fully or partially elasticized or may be foreshortened to provide a void space between the top sheet 18 and the absorbent core 14. Exemplary structures including elasticized or foreshortened top sheets are described in more detail in U.S. Pat. No. 5,037,416 entitled “Disposable Absorbent Article Having Elastically Extensible Top sheet” issued to Allen et al. on Aug. 6, 1991; and U.S. Pat. No. 5,269,775 entitled “Trisection Top sheets for Disposable Absorbent Articles and Disposable Absorbent Articles Having Such Trisection Top sheets” issued to Freeland et al. on Dec. 14, 1993.

The back sheet 26 may be joined with the top sheet 18. The back sheet 20 may prevent the exudates absorbed by the absorbent core 14 and contained within the diaper 10 from soiling other external articles that may contact the diaper 10, such as bed sheets and undergarments. In certain embodiments, the back sheet 26 may be substantially impervious to liquids (e.g., urine) and comprise a laminate of a nonwoven and a thin plastic film such as a thermoplastic film having a thickness of about 0.012 mm (0.5 mil) to about 0.051 mm (2.0 mils). Suitable back sheet films include those manufactured by Tredegar Industries Inc. of Terre Haute, Ind. and sold under the trade names X15306, X10962, and X10964. Other suitable back sheet materials may include breathable materials that permit vapors to escape from the diaper 10 while still preventing exudates from passing through the back sheet 10. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont. Other breathable back sheets including nonwoven webs and apertured formed films are described in U.S. Pat. No. 5,571,096 issued to Dobrin et al. on Nov. 5, 1996.

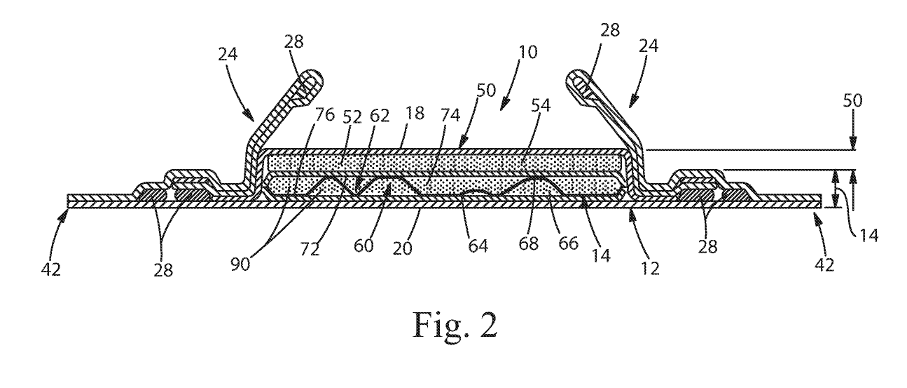

FIG. 2 shows a cross section of FIG. 1 taken along the sectional line 2-2 of FIG. 1. Starting from the wearer facing side, the diaper 10 may comprise the top sheet 18, the components of the absorbent core 14, and the back sheet 20. According to a certain embodiment, diaper 10 may also comprise an acquisition system 50 disposed between the liquid permeable top sheet 18 and a wearer facing side of the absorbent core 14. The acquisition system 50 may be in direct contact with the absorbent core. The acquisition system 50 may comprise a single layer or multiple layers, such as an upper acquisition layer 52 facing towards the wearer's skin and a lower acquisition 54 layer facing the garment of the wearer. According to a certain embodiment, the acquisition system 50 may function to receive a surge of liquid, such as a gush of urine, and quickly absorb the liquid and distribute it across the absorbent core 14 so that the absorbent core absorbs the liquid before the liquid flows beyond the absorbent layer 14 and out of the diaper 10. In other words, the acquisition system 50 may serve as a temporary reservoir for liquid until the absorbent core 14 can absorb the liquid.

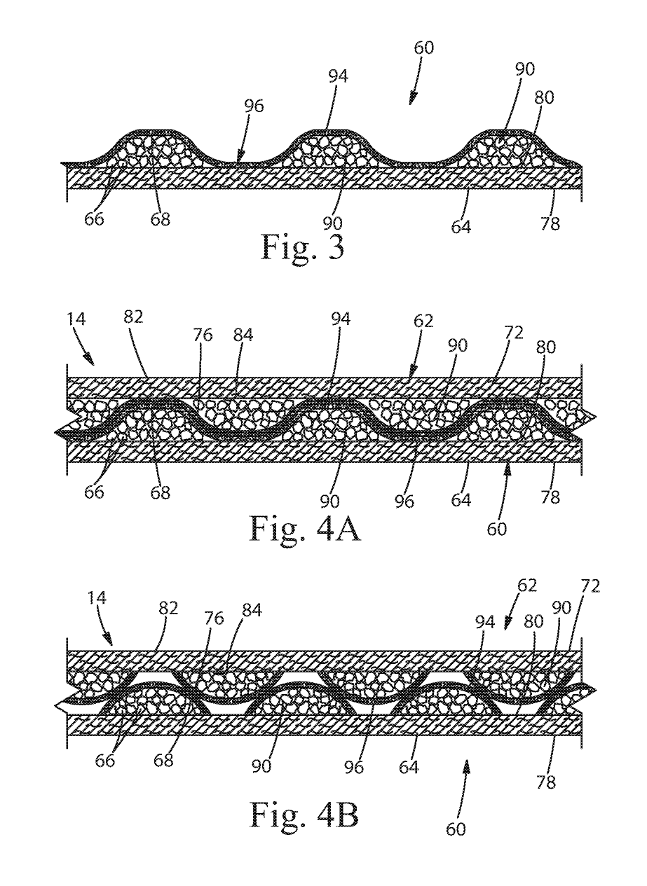

The absorbent core 14 is disposed between the top sheet 18 and the back sheet 20 and comprises two layers, a first absorbent layer 60 and a second absorbent layer 62. As shown in FIG. 3, the first absorbent layer 60 of the absorbent core 14 comprises a substrate 64, an absorbent particular polymer material 66 on the substrate 64, and a thermoplastic composition 68 on the absorbent particulate polymer material 66 and at least portions of the first substrate 64 as an adhesive for covering and immobilizing the absorbent particulate polymer material 66 on the first substrate 64. According to another embodiment, the first absorbent layer 60 of the absorbent core 14 may also include a cover layer on the thermoplastic composition 68.

Likewise, as illustrated in FIG. 2, the second absorbent layer 62 of the absorbent core 14 may also include a substrate 72, an absorbent particulate polymer material 74 on the second substrate 72, and a thermoplastic composition 66 on the absorbent particulate polymer material 74 and at least a portion of the second substrate 72 for immobilizing the absorbent particulate polymer material 74 on the second substrate 72. Although not illustrated, the second absorbent layer 62 may also include a cover layer such as the cover layer.

The substrate 64 of the first absorbent layer 60 may be referred to as a dusting layer and has a first surface 78 which faces the back sheet 20 of the diaper 10 and a second surface 80 which faces the absorbent particulate polymer material 66. Likewise, the substrate 72 of the second absorbent layer 62 may be referred to as a core cover and has a first surface 82 facing the top sheet 18 of the diaper 10 and a second surface 84 facing the absorbent particulate polymer material 74. The first and second substrates 64 and 72 may be adhered to one another with adhesive about the periphery to form an envelope about the absorbent particulate polymer materials 66 and 74 to hold the absorbent particulate polymer material 66 and 74 within the absorbent core 14.

According to a certain embodiment, the substrates 64 and 72 of the first and second absorbent layers 60 and 62 may be a non-woven material. In certain embodiments, the non-wovens are porous and in one embodiment has a pore size of about 32 microns.

As illustrated in FIGS. 3, 4a, and 4b, the absorbent particulate polymer material 66 and 74 is deposited on the respective substrates 64 and 72 of the first and second absorbent layers 60 and 62 in clusters 90 of particles to form a grid pattern comprising land areas 94 and junction areas 96 between the land areas 94. The junction areas 96 in the grid pattern contain little or no absorbent particulate polymer material 66 and 74. The land areas 94 and junction areas 96 can have a variety of shapes including, but not limited to, circular, oval, square, rectangular, triangular, and the like.

The first and second absorbent layers 60 and 62 may be combined together to form the absorbent core 14 such that the grid patterns of the respective first and second absorbent layers 62 and 64 are offset from one another along the length and/or width of the absorbent core 14. The respective grid patterns may be offset such that the absorbent particulate polymer material 66 and 74 is substantially continuously distributed across the absorbent particulate polymer area. In a certain embodiment, absorbent particulate polymer material 66 and 74 is substantially continuously distributed across the absorbent particulate polymer material area despite the individual grid patterns comprising absorbent particulate polymer material 66 and 74 discontinuously distributed across the first and second substrates 64 and 72 in clusters. In a certain embodiment, the grid patterns may be offset such that the land areas 94 of the first absorbent layer 60 face the junction areas 96 of the second absorbent layer 62 and the land areas 94 of the second absorbent layer 62 face the junction areas 96 of the first absorbent layer 60. When the land areas 94 and junction areas 96 are appropriately sized and arranged, the resulting combination of absorbent particulate polymer material 66 and 74 is a substantially continuous layer of absorbent particular polymer material across the absorbent particulate polymer material area of the absorbent core 14. In a certain embodiment, respective grid patterns of the first and second absorbent layer 60 and 62 may be substantially the same.

In a certain embodiment, the amount of absorbent particulate polymer material 66 and 74 may vary along the length of the grid pattern. In a certain embodiment, the grid pattern may be divided into any number of zones, in which the amount of absorbent particulate polymer material 66 and 74 varies from zone to zone. The amount of absorbent particulate polymer material 66 and 74 may, in a certain embodiment, gradually transition from one of the plurality of absorbent zones to another. This gradual transition in amount of absorbent particulate polymer material 66 and 74 may reduce the possibility of cracks forming in the absorbent core 14.

The amount of absorbent particulate polymer material 66 and 74 present in the absorbent core 14 may vary, but in certain embodiments, is present in the absorbent core in an amount greater than about 80% by weight of the absorbent core, or greater than about 85% by weight of the absorbent core, or greater than about 90% by weight of the absorbent core, or greater than about 95% by weight of the core. In a particular embodiment, the absorbent core 14 consists essentially of the first and second substrates 64 and 72, the absorbent particulate polymer material 66 and 74, and the thermoplastic adhesive composition 68 and 76. In an embodiment, the absorbent core 14 may be substantially cellulose free.

In certain embodiments which are not substantially cellulose free, the absorbent core 14 can include some amount of cellulose fiber material, such as airfelt. A relatively low amount of cellulosic material is used, in certain embodiments, less than 40 weight percent, or 20 weight percent of cellulosic material, as compared to the weight of absorbent core.

Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,207 (Dyer et al.); U.S. Pat. No. 5,397,316 (LaVon et al.); and U.S. Pat. No. 5,625,222 (DesMarais et al.).

The thermoplastic material 68 and 76 may serve to cover and at least partially immobilize the absorbent particulate polymer material 66 and 74. In one embodiment of the present invention, the thermoplastic material 68 and 76 can be disposed essentially uniformly within the absorbent particulate polymer material 66 and 74. However, in a certain embodiment, the thermoplastic material 68 and 76 may be provided as a fibrous layer which is at least partially in contact with the absorbent particulate polymer material 66 and 74 and partially in contact with the substrate layers 64 and 72 of the first and second absorbent layers 60 and 62. FIGS. 3, 4a, and 4b show such a structure, and in that structure, the absorbent particulate polymer material 66 and 74 is provided as a discontinuous layer, and a layer of fibrous thermoplastic material 68 and 76 is laid down onto the layer of absorbent particulate polymer material 66 and 74, such that the thermoplastic material 68 and 76 is in direct contact with the absorbent particulate polymer material 66 and 74, but also in direct contact with the second surfaces 80 and 84 of the substrates 64 and 72, where the substrates are not covered by the absorbent particulate polymer material 66 and 74. This imparts an essentially three-dimensional structure to the fibrous layer of thermoplastic material 68 and 76, which in itself is essentially a two-dimensional structure of relatively small thickness, as compared to the extension in length and width directions. In other words, the thermoplastic material 68 and 76 undulates between the absorbent particulate polymer material 68 and 76 and the second surfaces of the substrates 64 and 72.

According to certain embodiments, the thermoplastic material 68 and 76 can comprise any thermoplastic material, including, but not limited to adhesive thermoplastic materials, also referred to as hot melt adhesives. Some initially thermoplastic materials may later lose their thermoplasticity due to a curing step, e.g., initiated via heat, UV radiation, electron beam exposure or moisture or other means of curing, leading to the irreversible formation of a crosslinked network of covalent bonds. Those materials having lost their initial thermoplastic behavior are herein also understood as thermoplastic materials.

Alternatively, the absorbent core of the present invention may include only one layer. In such an embodiment, the absorbent core includes a first absorbent layer, the first absorbent layer including a first substrate, absorbent particulate polymer material deposited on the first substrate, and thermoplastic material covering the absorbent particulate polymer material on the first substrate, the absorbent particulate polymer material is substantially continuously distributed across the absorbent particulate polymer material area. Alternatively, the first absorbent layer may include a second substrate.

### Ink Composition

An article of the present invention may comprise a reactive ink composition 150. The ink compositions of the present invention show benefits in the areas of CIE Lab Delta E between ink printed and garment-facing surfaces, ink penetration, ink adhesion and leachability. Oftentimes, pigments tend to solubilize when exposed to certain chemical substances. This is known as “leachability” and may also be referred to as “pigment bleed.” In diaper usage, many current printing inks may react with or be leached by common fluids such as water, saline solution, baby oil, etc. that may come into contact with a baby's skin. These fluids may cause the ink-printed image to rub off, smear, transfer to the skin, transfer to furniture and/or other surfaces.

Printing on substrates can be challenging, particularly when the substrate surface is not uniform, flat or consistent. Nonwovens are fibrous webs which are bonded by a variety of means according to their method of manufacture. Being able to print an image on a nonwoven substrate and reproduce the desired colors with existing printing processes may be a challenge. When the image is printed on a nonwoven on the surface opposite to the surface facing the garment or opposite to the surface which will be viewed by the user of the article, the colors on the image may be typically distorted by the inherent color and opacity of the nonwoven. This poses a problem as color will be different from the intended color of the image. One solution is to print the image with an ink which achieves a high ink penetration on the nonwoven beyond the outermost fibers on the printed surface. The ink composition of the present invention has a high ink penetration which results in vibrancy of the colors on the garment facing surface.

Embodiments of the present invention include an ink composition comprising a reactive ink. The reactive ink composition may include a binder component and a crosslinking agent. Ink compositions useful in the present invention are manufactured by Resino Trykfarver AS (Ballerup, Denmark) with trade names of REDIVERS 300 Promoter 5032 and RETURIN 194-50 for the crosslinking additive and ink system respectively. The ink composition of the present invention is manufactured by Resino and is commercially available from Innowo Print AG (Ilsenburg, Germany).

The ink composition of the present invention includes a binder. The binder may be present in an amount of from about 5% to about 20%, from about 8% to about 14% of the total solids composition. Total solids composition is the ink components which remain on the substrate after drying. The binder may be selected from the group consisting of nitrocellulose, polyamides, polyurethanes, acrylic dispersions, rosin resins, ketone and polyketone resin, polyvinyl butyral, polyvinyl acetate, polyethylene imide, modified celluloses including ethyl cellulose, cellulose esters, cellulose acetate propionate, and cellulose acetate butyrate, polyvinyl alcohol, maleic resin, polyester resin, and mixtures thereof. In one embodiment, the binder is a mixture of polyvinyl butyral and nitrocellulose.

In one embodiment, the reactive ink composition may comprise a crosslinking additive. The crosslinker may be present in an amount of from about 0.1% to about 10%, from about 0.3% to about 6%, from about 0.5% to about 6% of the total solids content. The crosslinker may be selected from the group consisting of poly-isocyanates, blocked poly-isocyanates, poly-epoxy compounds, poly-aziridines, carbodiimides, poly-silanes, and mixtures thereof.

The ink composition may also comprise adhesion promoters, including but not limited to, Zirconium compounds, Aluminum compounds, Zircoaluminates, Chlorinated polyolefins, Acrylates, silanes, and mixtures thereof.

The ink composition may also comprise additional components including, but not limited to, perfumes, tackifiers, fillers, waxes, viscosity modifiers, surfactants, wetting agents, slip agents, defoamers, pH adjusters, and mixtures thereof.

In one embodiment, the ink composition is organic solvent-based or non-aqueous based. In an alternative embodiment, the ink composition is water-based. In one embodiment, the ink composition does not comprise a water-based aliphatic urethane compound.

The ink composition may be applied to the substrate by any method known in the art. Specifically, the ink composition may be applied to the substrate using ink jet printers, flexographic printing presses, gravure printing presses, offset printing press, screen printing methods or a combination thereof. The ink composition may be printed on a number of article components including, but not limited to, the backsheet, topsheet, cuffs, fastening tapes, wrappers, or any part of the article, or primary or secondary packaging. In one embodiment, the ink composition is applied to the substrate by flexographic or rotogravure printing. A metering roll or doctor blade system may be used. In one embodiment, printing is performed in excess of 150 m/min.

The ink printed substrate may be a nonwoven substrate. The nonwoven substrate may have a basis weight of from about 8 gsm to about 65 gsm; from about 8 gsm to about 45 gsm; from about 10 gsm to about 35 gsm; from about 15 gsm to about 25 gsm. The nonwoven substrate may have an ink printed inner side and a non-printed garment facing side. The reactive ink composition may be printed on the nonwoven substrate on the ink printed inner side, forming an ink printed area. Alternatively, the nonwoven substrate may have an ink printed garment facing side and a non-printed inner side. The printed nonwoven substrate may be combined with a film and/or a second nonwoven substrate after printing to form a laminate such that the ink printed inner side is in contact with the film and/or the second nonwoven. Alternatively, the printed nonwoven substrate may be combined with a film and/or a second nonwoven substrate after printing to form a laminate such that the non-printed inner side is in contact with the film and/or the second nonwoven. In still a further nonlimiting example, nonwoven substrate may be folded after printing such that the ink printed inner side is sandwiched between sections of the nonwoven substrate.

In some embodiments, prior to printing the ink composition on the substrate, the substrate may undergo a surface treatment, including but not limited to corona treatment. Corona treatment is a method of increasing surface energy on substrates to promote surface wetting of and adhesion to the substrate when printing, coating or laminating. The purpose of corona treatment is to increase the surface energy of the substrate web to improve the wettability and adhesion characteristics of inks and adhesives to polyolefin films and nonwoven substrates. Unlike the purely mechanical bond, (as in the case of an ink penetrating into a porous surface like paper), plastic films and nonwoven substrates may need some means of surface treatment to achieve acceptable chemical bonding with the ink or adhesive.

Corona treatment systems are made of several components designed to apply a high voltage, high frequency electrical discharge to the substrate. Corona discharge introduces polar groups into the polymeric surfaces and, as a consequence, increases its surface energy, wettability, and adhesion characteristics. The main chemical mechanism of corona treatment is oxidation. The high voltage ionizes the air in the air gap creating a corona, which modifies the surface and increases the surface energy of the substrate passing over the electrically grounded roll. The effect is not long lasting and the increase in the surface energy dissipates within two to six weeks. Corona treatments may be applied at from about 1.0 to about 18.0 watts per square meters per minute; from about 2.5 to about 15.0 watts per square meters per minute; or about 7.5 watts per square meters per minute.

Thus, in some embodiments of the present invention, by utilizing surface treatments such as corona treatment mechanisms, the surface energy of substrates may be increased, thereby improving adhesion of ink to a substrate.

### Test Measurement Methods

### Method for Measuring Print Color and Print Density

Print color and density on a printed nonwoven or film is measured using a hand held, 45°/0° configuration, hemispherical geometry spectrophotometer, the X-rite eXact Spectrophotometer (available from X-Rite, Grand Rapids Mich.), or equivalent instrument, with a 4.0 mm optical aperture. This instrument measures print density based on reflection density expressed as the logarithm of the reciprocal of the reflectance factor. Set the scale to L*a*b* units, 2° Observer, C Illumination, Abs White Base, no Physical Filter, and the Density Standard of ANSI T. Measurements are performed in an environment controlled lab held at about 23° C.±2 C.° and 50%±2% relative humidity.

Calibrate the instrument per the vendor's instructions using the standard white board (available as PG2000 from Sun Chemical-Vivitek Division, Charlotte, N.C.) (or alternatively a white ceramic plate (Available from Hunter Associates, CIELab values of approximately L*=93.6, a*=−1.3, b*=0.6)) each day before analyses are performed. Remove the substrate to be measured from the sample article. If necessary, a cryogenic freeze-spray (e.g., Cyto-freeze, available from Control Company, Houston Tex.) can be used to facilitate removal. Samples are conditioned at about 23° C.±2 C.° and 50%±2% relative humidity for 2 hours before testing.

Place the Standard White Board on a horizontal bench, standard side facing upward. Place the specimen flat on top of the Standard White Board with the printed side facing upward. Place the eXact spectrophotometer on the specimen such that the measurement site is free of folds and wrinkles and 100% of the measurement site is within the instrument's aperture. Take a reading for density and L*a*b* color and record each to the nearest 0.01 units.

In like fashion the measure is repeated on corresponding sites on five (5) substantially similar printed substrates and the density and L*a*b* color values averaged separately and reported to the nearest 0.01 units.

There are various calculations used to evaluate how much a color deviates from an intended color. Two calculations widely accepted in the industry are Delta E* (CIE 1976) and Delta E (CMC 2:1). The Delta E* (CIE 1976) between two colors with measured L*₁a*₁b*₁ and L*₂a*₂b*₂ values is calculated as follows:

ΔE_{CMC}*=\sqrt{(L*₁−L*₂)²+(a*₁−a*₂)²+(b*₁−b*₂)²}

Delta E (CMC 2:1) is a color tolerancing system which takes into consideration that the eye is more sensitive to chroma than lightness. Chroma characterizes how vivid (saturated) or dull (gray) a color is. Lightness represents the luminous intensity of a color. This calculation is widely used in the textiles industry. The Delta E (CMC 2:1) between two colors with measured L₁*a₁*b₁* and L₂*a₂*b₂* values is calculated as follows:

ΔE_{CMC}=\sqrt{[ΔL/(l·S_{L})]²+[ΔC*/(c·S_{c})]²+(ΔH*/S_{H})²}

Where:
ΔL*=L*₁−L^{*}₂
l=lightness weight. A value of 2.0 is commonly used herein per ASTM D2244-11.

$${S}_{L} = \{ \begin{matrix}0.511 , & {L}_{1}^{*} < 16 \\ \frac{0.040975 \cdot {L}_{1}^{*}}{1 + 0.01765 \cdot {L}_{1}^{*}} , & {L}_{1}^{*} \geq 16\end{matrix}$$

C*₁=\sqrt{a*₁²+b_{*1}²}
C*₂=\sqrt{a*₂²+b*₂²}
ΔC*=C*₁−C*₂
c=chroma weight. A value of 1.0 is commonly used herein per ASTM D2244-11.

$${S}_{C} = \frac{0.0638 \cdot {C}_{1}^{*}}{1 + 0.0131 \cdot {C}_{1}^{*}} + 0.638$$

ΔH*=s\sqrt{2(C*₁−C*₂−a*₁·a*₂ b*₁·b*₂)}
   if a*₁·b*₂>a*₂·b*₁ then
   s=1
   else
   s=−1
   end if.
h₁=tan⁻¹(b*₁/a*₁)

$$T = \{ \begin{matrix}0.56 + \vert 0.2 cos ( {h}_{1} + 168 ° ) \vert , & 164 ° \leq {h}_{1} \leq 345 ° \\ 0.36 + \vert 0.4 cos ( {h}_{1} + 35 ° ) \vert , & otherwise\end{matrix}$$

f=\sqrt{(C*₁)⁴/[(C*₁)⁴+1900]}
S_{H}=S_{c}(f·T+1−f)
   Generally in the textiles industry color consistency is controlled within Delta E CMC values ranging from 3.0 to 6.0. The particular limit is set considering the textile surface, uniformity and characteristics.

### Ink Penetration Test Method

### Equipment:

Razor blade: VWR Surgical Carbon Steel, No. 9, Single Edge Industrial Blades, or equivalent.
Double Sided Conductive Carbon tape: SPI Supplies® Brand, 25 mm wide, or equivalent
Zeiss Discovery V20 Stereo-light microscope with Z-motorized stage, Carl Zeiss Microimaging GmbH, Göttingen, Germany.
MRc5 (5 MP, Color) Zeiss Camera, Carl Zeiss Microimaging GmbH, Gottingen, Germany.
Axiovision software version 4.8.3.0 with Z-stack & Extended Focus, Carl Zeiss Microimaging GmbH, Gottingen, Germany.

### Procedure

A subsample approximately 2 cm in length and about 1-2 cm in width is cut from the web region containing printed ink. With the print side facing down, the subsample is sectioned using a new razor blade, with a straight down motion, perpendicular to substrate. The sectioned subsample is adhered to an approximately 2 cm by 2 cm by 1 mm thick Al mount using double sided carbon tape. The double-sided carbon tape should extend from the front to the back of the mount, so that the mount edge that will contain the x-sectioned substrate edge, is completely covered before the substrate is attached (i.e. no Al exposed.) The sectioned substrate is then placed on top of the taped mount with printed side up and the sectioned edge at a mount edge. The mount is tilted and securely held at 90° so that images of the x-sectioned substrate face can be acquired.

The cross-section is viewed with reflected halogen light using a Zeiss Discovery V20 Stereo-light microscope equipped with a Z-motorized stage and MRc5 (5 MP, Color) Zeiss Camera at −80× magnification. The microscope is interfaced with Axiovision software version 4.8.3.0 with Z-stack & Extended Focus modules. Images of the cross-section are collected using a Z-stack module of the Axiovision software, then processed using Extended Focus module of the Axiovision software (wavelets method) to create a 2-D representation of the cross-section. The Z-stack range is chosen in order to bring the cross-sectional plane into focus where a typical range is about 5-50 μm and the step size is typically 1-5 μm.

The ink penetration depth is measured in microns from the top surface of the nonwoven to the distance where ink can no longer be observed by image analysis software. The top surface is defined as the uppermost exposed area comprising printed ink.

This distance is measured with a calibrated manual line tool in image analysis software, such as Axiovision, and reported as the ink penetration depth.

Ink penetration depth is measured in a fiber region deposed between two bonding sites on the nonwoven. The percent ink penetration for a given fiber region is determined by dividing the ink penetration depth for said fiber region by the maximum nonwoven thickness for the same fiber region. The maximum nonwoven thickness is the maximum distance between opposing sides of the nonwoven in the z-direction (i.e., the maximum distance between the top and bottom surfaces of the nonwoven).

### Leachability of Printed Substrates

Leachability of a printed is determined by soaking a printed nonwoven in various test fluids at elevated for 12 hours and evaluated by visible spectroscopy. A spectrophotometer capable of scanning between 350 nm and 800 nm using a 1 cm cuvette (a suitable instrument is a Perkin Elmer UV-VIS spectrometer Lambda 2 available from PerkinElmer GmbH, or equivalent). The test fluids are 0.9% NaCl in water (saline), 99+% isopropanol, and clear baby oil (a suitable product is WELL BEGINNINGS baby oil, distributed by Walgreen Co. of Deerfield Ill., or the equivalent).

Remove a printed nonwoven sample from 27 replicate absorbent articles, using freeze spray (such as Cyto-Freeze, Control Company, Houston Tex.) as necessary to remove the layer without distorting the dimensions of the nonwoven or damaging the printing. Visually locate a high print density region. Cut a specimen 60 mm×60 mm at that target site and at an equivalent site from each of the other articles. Place the specimens, three per vial into a total of nine 20 mL screw-top vials. Label three vials, one set each, for with each of the three test fluids.

Pipet 15.00 mL of saline into 3 vials, 15.00 mL of isopropanol into 3 vials, and 15.00 mL of baby oil into the last three vials. Cap each vial and shake to wet and submerge the specimens. Place all vials into an laboratory oven maintained a 37° C.±2 C.° for 12 hours. Afterwards remove the vials and allow them to cool to room temperature.

Set up and calibrate the spectrophotometer per the vendor's instructions. Set the instrument to scan from 350 nm to 800 nm. An optically clear glass cuvette with 1.00 cm path length is used for measurements. Collect a spectrum from an aliquot from each vial. Rinse the cuvette between each measure. Determine the maximum absorbance of each spectrum. Calculate the average of the maximum absorbance for each set of replicates (3) for all test fluids, and report separately to the nearest 0.001 absorbance units.

### Ink Rub-Off

Ink rub-off of a nonwoven substrate is performed on a Gakushin-type Rubbing Tester (a suitable instrument is a Model RT-300 available from Daiei Kagaku Seiki, Kyoto Japan or equivalent). The test specimen is mounted on the instrument and is rubbed against a standard abrading surface consisting of a receptor swatch (76.2 mm×25.4 mm, standard Crock Cloth Style #3, or equivalent) and a Standard Nonwoven (76.2 mm×25.4 mm, 13.5 gsm standard spunbond nonwoven, type 900-SB050, or equivalent) both available from Testfabrics Inc, West Piston, Pa. The Receptor Swatch is then measured using a spectrophotometer capable of making CIE L*a*b* measurements (a suitable instrument is the X-Rite eXact available from X-Rite, Grande Rapids, Mich. with 4 mm aperture, or equivalent) and the Ink Adhesion Rating (IAR) is calculated. All testing is performed at about 23° C.±2 C.° and a relative humidity of about 50%±2%.

Specimens are tested using the following test fluids: a mineral oil having a Bp of 215° C.-643° C., flash point of 115° C. to 268° C., Density of 0.82 to 0.90 g/cm³, and dynamic viscosity of 0.038 Pa. s at 38° C. (a suitable oil is RC-118 available from G-Biosciences, St. Louis, Mo. or equivalent) and a diaper rash cream containing zinc oxide and oil with a viscosity >10 Pa. s and shear rate of 10 sec⁻¹ at 25° C. Suitable diaper rash creams include HIPOGLOS® (available from Andromaco Laboratories of Buenos Aires, Argentina) DESITIN® Maximum Strength (manufactured by Johnson & Johnson of New Brunswick, N.J.), A+D® Diaper Rash Cream (manufactured by from Bayer AG of Germany) or equivalent.

All samples and test fluids are conditioned at about 23° C.±2 C.° and a relative humidity of about 50%±2% for two hours prior to analysis. Remove a printed nonwoven specimen from the absorbent article, using freeze spray (such as Cyto-Freeze, Control Company, Houston Tex.) as necessary to remove the layer without distorting the dimensions of the nonwoven or damaging the printing. Visually locate the region on the sample with the highest print density. Cut a specimen 230 mm×25.4 mm of the nonwoven to be tested. In like fashion harvest 4 more test specimens from the equivalent position on four (4) additional replicate articles. Mount the five test specimens onto the movable, curved test stage with the printed sides facing away from the stage. The test specimens must be secured so that they do not move during testing. If a nonwoven specimen is not durable enough for rub testing (e.g. will stretch or tear), it is laminated onto a 300 mm by 25.4 mm sheet of copy paper with 2-sided tape before testing. In addition to the test specimens, a Control Nonwoven is also analyzed for comparison and calculation of ΔE_{CMC}. The Control Nonwoven is compositionally identical to the specimen nonwoven with the exception that it has no printing within the 300 mm by 25.4 mm strip. If a non-printed area of the nonwoven is not available the Standard Nonwoven can be used as the Control Nonwoven.

For rub tests utilizing the mineral oil test fluid, place a Receptor Swatch on a bench and stack a Standard Nonwoven overlying it. Mount the stack on to the friction heads of the Rubbing Tester with the Standard Nonwoven facing outward and secure with the receptor clamp. Accurately pipet 0.25 mL of mineral oil onto the surface of the stack, allowing it to soak in. Lower the friction heads down onto the test specimen such that the Standard Nonwoven is in contact with the test specimens. Set the instrument to perform 15 cycles and start the test. Once complete, remove the Receptor Swatch and Standard Nonwoven from the friction, and discard the Standard Nonwoven. Allow the Receptor Swatch to condition for 24 hours at about 23° C.±2 C.° and a relative humidity of about 50%±2% before evaluating.

For rub tests utilizing the diaper cream, place the Receptor Swatch onto the bench. Diaper Cream is applied in a specific pattern using a template. The template is a piece of plastic 76.2 mm by 25.4 mm by 0.60 mm thick into which 9 holes, each 6 mm in diameter, are drilled 3 to a row in a region approximately 20 mm by 20 mm square at the lateral and longitudinal center of the template. Place the template over the Receptor Swatch. Fill the holes flush with cream and remove the template. Stack the Standard Nonwoven on top of the Receptor Swatch. Mount the stack on to the friction heads of the Rubbing Tester with the Standard Nonwoven facing outward and secure with the receptor clamp. Lower the friction heads down onto the test specimen such that the Standard Nonwoven is in contact with the test specimens. Set the instrument to perform 15 cycles and start the test. Once complete, remove the Receptor Swatch and Standard Nonwoven from the friction head. Do not discard the Standard Nonwoven it will be analyzed as the Receptor Swatch/Standard Nonwoven stack. Allow the Receptor Swatch/Standard Nonwoven stack to condition for 24 hours at about 23° C.±2 C.° and a relative humidity of about 50%±2% before evaluating.

Calibrate the spectrophotometer as per the manufacturer's instructions. Set the spectrophotometer to Abs white base, No Physical filter, illuminant C, 2° observer, ANSI T Density Standard. Place the Receptor Swatch onto the white ceramic plate (Available from Hunter Associates, with CIE L*a*b* values of approximately L*=93.6, a*=−1.3, b*=0.6). Visually divide the 20 mm×20 mm rub-off region into four quadrants. Measure L*a*b* centered at the most visually intense site within each quadrant, and then at the center of the rub-off region. Record values for the Control and each of the other specimens. Average the five readings for each and calculate the ΔE_{(CMC)} of the specimens verses the Control. Repeat for all mineral oil and diaper cream samples. Report as the arithmetic mean of ΔE_{cmc} of the 5 replicates to the nearest 0.01, separately for both mineral oil and diaper cream. Calculate and report Ink Adhesion Rating (IAR) for each of the test fluids using the following equation:

$$IAR = 5.04 {( 1 + 0.47 ( \frac{Δ {E}_{CMC}}{14.70} ) )}^{- \frac{1}{0.47}} .$$

### EXAMPLES

Examples 1-9 below are illustrative embodiments of the present invention. Comparative examples 1-6 are structures known in the art. The examples and comparative examples are tested in accordance with test methods described herein.

### Example 1

Example 1 is a printed nonwoven. The nonwoven is a bicomponent polyethylene/polypropylene spunbond nonwoven and has basis weight of 17 gsm. The ink composition is 100% Process Magenta Resino RETURIN 194-50 with REDIVERS 300 Promoter 5032, the latter being a crosslinking additive. The ink is printed onto the ink printed surface using flexographic printing.

### Example 2

Example 2 is a printed nonwoven. The nonwoven is a bicomponent polyethylene/polypropylene spunbond nonwoven and has basis weight of 17 gsm. The ink composition is 100% Process Cyan Resino RETURIN 194-50 ink with REDIVERS 300 Promoter 5032, the latter being a cross-linking additive. The ink is printed onto the ink printed surface using flexographic printing.

### Example 3

Example 3 is a printed nonwoven. The nonwoven is a bicomponent polyethylene/polypropylene spunbond nonwoven and has basis weight of 17 gsm, commercially available from Fibertex Nonwovens of Aalborg, Denmark. The ink composition is 100% Process Cyan Resino RETURIN 194-50 ink with REDIVERS 300 Promoter 5032, the latter being a cross-linking additive. The ink is printed onto the ink printed surface using flexographic printing.

### Example 4

Example 4 is a printed nonwoven. The nonwoven is a bicomponent polyethylene/polypropylene spunbond nonwoven and has basis weight of 17 gsm, commercially available from Fibertex Nonwovens of Aalborg, Denmark. The ink composition is 75% PMS 218 Resino RETURIN 194-50 ink with REDIVERS 300 Promoter 5032, the latter being a cross-linking additive. The ink is printed onto the ink printed surface using flexographic printing.

### Example 5

Example 5 is a printed nonwoven. The nonwoven is a bicomponent polyethylene/polypropylene spunbond nonwoven and has basis weight of 17 gsm, commercially available from Fibertex Nonwovens of Aalborg, Denmark. The ink composition is 100% PMS 3005 Resino RETURIN 194-50 ink with REDIVERS 300 Promoter 5032, the latter being a cross-linking additive. The ink is printed onto the ink printed surface using flexographic printing.

### Example 6

Example 6 is a printed nonwoven. The nonwoven is a bicomponent polyethylene/polypropylene spunbond nonwoven and has basis weight of 17 gsm, commercially available from Gulsan of Gaziantep, Turkey. The ink composition is 100% Process Cyan Resino RETURIN 194-50 ink with REDIVERS 300 Promoter 5032, the latter being a cross-linking additive. The ink is printed onto the ink printed surface using flexographic printing after the substrate has been treated with corona treatment at various levels specified in this filing.

### Example 7

Example 7 is a printed nonwoven. The nonwoven is a bicomponent polyethylene/polypropylene spunbond nonwoven and has basis weight of 17 gsm, commercially available from Gulsan of Gaziantep, Turkey. The ink composition is 100% Process Cyan Resino RETURIN 194-50 ink with REDIVERS 300 Promoter 5032, the latter being a cross-linking additive. The ink is printed onto the ink printed surface using flexographic printing after the substrate has been treated with corona treatment 2.5 W/m²/min. A reactive lacquer is applied over the ink printed surface at the flexographic printing press in such manner that the lacquer covers the entirety of the ink printed surface. The reactive lacquer composition is Resino RETURIN 194-50 ink that is void of pigment and REDIVERS 300 Promoter 5032.

### Example 8

Example 8 is a printed nonwoven. The nonwoven is a bicomponent polyethylene/polypropylene spunbond nonwoven and has basis weight of 17 gsm, commercially available from Gulsan of Gaziantep, Turkey. The ink composition is 100% Process Cyan Resino RETURIN 194-50 ink with REDIVERS 300 Promoter 5032, the latter being a cross-linking additive. The ink is printed onto the ink printed surface using flexographic printing after the substrate has been treated with corona treatment 7.5 W/m²/min. A reactive lacquer is applied over the ink printed surface at the flexographic printing press in such manner that the lacquer covers the entirety of the ink printed surface. The reactive lacquer composition is Resino RETURIN 194-50 ink that is void of pigment and REDIVERS 300 Promoter 5032.

### Example 9

Example 9 is a printed nonwoven. The nonwoven is a bicomponent polyethylene/polypropylene spunbond nonwoven and has basis weight of 17 gsm, commercially available from Gulsan of Gaziantep, Turkey. The ink composition is 100% Process Cyan Resino RETURIN 194-50 ink with REDIVERS 300 Promoter 5032, the latter being a cross-linking additive. The ink is printed onto the ink printed surface using flexographic printing. The substrate was not corona treated and is void of reactive lacquer.

### Comparative Example 1

Comparative Example 1 is a printed nonwoven landing zone taken from a Size 2 PAMPERS® diaper marketed by The Procter & Gamble Company in Germany from January 2013 to January 2014. The nonwoven has a basis weight of 45 gsm. Ink was printed onto the ink printed surface using flexographic printing. The ink composition does not include a cross-linking additive. Color measurements and ink penetration measurements below are measured from 100% Cyan color control block printed thereon.

### Comparative Example 2

Comparative Example 2 is a printed nonwoven landing zone taken from a Size 2 PAMPERS® diaper marketed by The Procter & Gamble Company in Germany from January 2013 to January 2014. The nonwoven has a basis weight of 45 gsm. Ink was printed onto the ink printed surface using flexographic printing. The ink composition does not include a cross-linking additive. Color measurements and ink penetration measurements below are measured from the green star printed thereon.

### Comparative Example 3

Comparative Example 3 is a printed nonwoven landing zone taken from a Size 2 PAMPERS® diaper marketed by The Procter & Gamble Company in Germany from January 2013 to January 2014. The nonwoven has a basis weight of 45 gsm. Ink was printed onto the ink printed surface using flexographic printing. The ink composition does not include a cross-linking additive. Color measurements and ink penetration measurements below are measured from the red star printed thereon.

### Comparative Example 4

Comparative Example 4 is a printed nonwoven landing zone taken from a Size 2 PAMPERS® diaper marketed by The Procter & Gamble Company in Germany from January 2013 to January 2014. The nonwoven has a basis weight of 45 gsm. Ink was printed onto the ink printed surface using flexographic printing. The ink composition does not include a cross-linking additive. Leachability measurements below are measured from the blue background printed thereon.

### Comparative Example 5

Comparative Example 5 is a printed film taken from the back sheet of a PAMPERS® CRUISERS® diaper marketed by The Procter & Gamble Company in North America from July 2011 to July 2012. The nonwoven has a basis weight of 16 gsm. Ink was printed onto the ink printed surface using flexographic printing. The ink composition does not include a cross-linking additive. Leachability measurements below are measured from the blue character face (COOKIE MONSTER®) printed thereon.

### Comparative Example 6

Comparative Example 6 is a printed film taken from the back sheet of a PAMPERS® CRUISERS® diaper marketed by The Procter & Gamble Company in North America from July 2011 to July 2012. The nonwoven has a basis weight of 16 gsm. Ink was printed onto the ink printed surface using flexographic printing. The ink composition does not include a cross-linking additive. Leachability measurements below are measured from the red character face (ELMO®) printed thereon.

### Data and Analysis

Table 1 below shows the L*a*b values taken from the ink printed surface and from the garment facing surface. The garment facing surface does not have ink directly printed thereon. However, as can be seen from the data below, the invention minimizes perceptible differences in color between the printed inner surface and the garment-facing surface.

TABLE 1
Print Color and Print Density

| | | | CIE Lab Delta | CMC Delta E |
|---|---|---|---|---|
| | | | E between Ink | between Ink |
| | Measurement taken | | and Garment | and Garment |
| | from | L*a*b | Surfaces | Surfaces |
| Example 1 | Ink Surface | 78.03, 31.05, −7.17 | 1.15 | 0.63 |
| | Gament Facing | 77.55, 30.47, −6.30 | | |
| | Surface | | | |
| Example 2 | Ink Surface | 78.66, −14.91, −23.41 | 1.53 | 0.93 |
| | Gament Facing | 77.71, −14.16, −24.08 | | |
| | Surface | | | |
| Comparative | Ink Surface | 70.92, −12.29, −22.14 | 7.53 | 4.13 |
| Example 1 | Garment Facing | 73.46, −7.56, −16.87 | | |
| | Surface | | | |
| Comparative | Ink Surface | 79.52, −17.91, 26.55 | 6.53 | 3.17 |
| Example 2 | Garment Facing | 77.56, −16.19, 20.68 | | |
| | Surface | | | |
| Comparative | Ink Surface | 72.53, 14.90, 20.54 | 8.30 | 5.44 |
| Example 3 | Garment Facing | 74.00, 11.79, 12.99 | | |
| | Surface | | | |

### Ink Penetration Data

The following table provides ink penetration levels for fiber regions. Two ink penetration depth measurements were taken for each example material in each fiber region:

TABLE 2
Percent Ink Penetration

| | Ink | Nonwoven | % Ink |
|---|---|---|---|
| | Penetration | Thickness | Penetration |
| | Depth | of Fiber Region | on |
| | (microns) | (microns) | Fiber Region |
| Example 1 - Fiber | 103 | 289.4 | 35.6% |
| Region 1A | | | |
| Example 1 - Fiber | 107 | 289.4 | 37.0% |
| Region 1A | | | |
| Example 1 - Fiber | 80 | 215.7 | 37.0% |
| Region 1B | | | |
| Example 1 - Fiber | 105.5 | 215.7 | 48.9% |
| Region 1B | | | |
| Comparative Example 1 - | 93 | 880 | 10.6% |
| Fiber Region CE1A | | | |
| Comparative Example 1 - | 28 | 880 | 3.2% |
| Fiber Region CE1A | | | |
| Comparative Example 1 - | 166 | 762 | 21.8% |
| Fiber Region CE1B | | | |
| Comparative Example 1 - | 140 | 762 | 18.4% |
| Fiber Region CE1B | | | |

### Leachability Data:

Table 3 shows leachability values when the examples are tested with WELL BEGINNINGS baby oil (available from Walgreens Co.) as per the Leachability Test Method herein. In addition, Table 3 shows data from said examples when tested with Peneteck light mineral oil NF and hexadecane (available from Acros Chemicals of New Jersey) in accordance with the steps outlined in the Leachability Test Method, substituting hexadecane and mineral oil (respectively) for the baby oil. Because baby oils may vary from manufacturer to manufacturer, these component materials (i.e., mineral oil and hexadecane) common to all baby oils were tested. Hexadecane is a base component of mineral oil, and mineral oil is a subcomponent of baby oil. The data below indicates that the present invention does not leach to the same extent as prior art in the presence of these base component materials. Table 4 shows leachability values when the examples are tested with baby oil, saline solution and isopropanol per the Leachability Test Method herein.

TABLE 3
Leachability - Baby Oil, Mineral Oil, and Hexadecane

| | Average of the Maximum Absorbance (AU) (n = 3) | |
|---|---|---|
| Test Solution | Example 3 | Comparative Example 4 |
| Baby Oil | 0.001 | 0.022 |
| | (Std. Dev. 0.001) | (Std. Dev. 0.015) |
| Mineral Oil | 0 - Below detection level | 0.067 |
| | | (Std. Dev. 0.058) |
| Hexadecane | 0 - Below detection level | 0.108 (Std. Dev. 0.064) |

TABLE 4
Leachability - Isopropanol, Saline Solution, Baby Oil

| | Average of the Maximum Absorbance (AU) | | | | |
|---|---|---|---|---|---|
| | (n = 3) | | | | |
| Test | | | Comparative | Comparative | Comparative |
| Solution | Example 4 | Example 5 | Example 4 | Example 5 | Example 6 |
| Isopropanol | 0.117 | 0.119 | 0.175 | 0.130 | 0.229 |
| | (Std dev | (Std dev | (Std dev | (Std dev 0.012) | (Std dev |
| | 0.006) | 0.007) | 0.003) | | 0.009) |
| Saline Solution | 0 - Below | 0 - Below | 2.30 × 10⁻³ | 5.76 × 10⁻² | 0.130 |
| Solution | detection | detection | (Std dev | (Std dev 0.021) | (Std dev |
| | level | level | 0.001) | | 0.023) |
| Baby Oil | 0 - Below | 0 - Below | 9.43 × 10⁻² | 2.59 × 10⁻² (Std | 3.05 × 10⁻³ |
| | detection | detection | (Std dev | dev 0.009) | (Std dev |
| | level | level | 0.005) | | 0.011) |

### Ink Adhesion Ratings

Table 5 shows ink adhesion ratings for various examples tested with mineral oil and diaper rash cream in accordance with the Ink Rub-Off Test Method herein. As can be seen, the ink adhesion rating (i.e., the resistance to ink rub off) is increased as the corona treatment level is increased within the ranges identified herein. The addition of a reactive lacquer over the reactive ink can improve the resistance to rub off even at low corona treatment levels, as shown in Table 5. It is known that high level corona treatments (e.g., above 20 W/m²/min) can result in damage to a substrate. The combination of reactive lacquer with corona treatment reduces the risk of damage to the substrate while improving ink adhesion.

TABLE 5
Ink Adhesion Ratings (IAR)

| | | IAR in Diaper |
|---|---|---|
| | IAR | Rash Cream 1 |
| | in Mineral Oil | (HIPOGLOS ®) |
| Example 6 Corona Treated at | 3.57 | 2.14 |
| 1.25 W/m²/min | (Std Dev. 0.24) | (Std Dev. 0.33) |
| Example 6 Corona Treated at | 4.16 | 2.30 |
| 2.5 W/m²/min | (Std Dev. 0.47) | (Std Dev. 0.28) |
| Example 6 Corona Treated at | 4.34 | 2.42 |
| 5 W/m²/min | (Std Dev. 0.20) | (Std Dev. 0.28) |
| Example 6 Corona Treated at | 4.44 | 2.58 |
| 7.5 W/m²/min | (Std Dev. 0.23) | (Std Dev. 0.19) |
| Example 6 Corona Treated at | 4.56 | 2.48 |
| 10 W/m²/min | (Std Dev. 0.21) | (Std Dev. 0.34) |
| Example 7 Corona Treated at | 4.93 | 3.57 |
| 2.5 W/m²/min and a Reactive | (Std Dev. 0.03) | (Std Dev. 0.33) |
| Lacquer | | |
| Example 8 Corona Treated | 4.84 | 3.18 |
| at 7.5 W/m²/min | (Std Dev. 0.10) | (Std Dev. 0.18) |
| and a Reactive Lacquer | | |
| Example 9 No Corona Treatment, | 3.01 | 2.20 |
| No Reactive Lacquer | (Std Dev. 0.34) | (Std Dev. 0.34) |

As noted above, baby oils may vary from manufacturer to manufacturer. However, Table 6 shows that the ink adhesion performance will be improved despite the type of baby oil. This is shown by testing mineral oil, which is a major component of baby oils and exhibits nearly the same results as the exemplary baby oil. The data in Table 6 relating to baby oil was obtained by following the steps outlined in the Ink Rub-Off Test Method and substituting Well Beginnings baby oil (available from Walgreens Co.) in lieu of mineral oil.

| | | IAR in Walgreens |
|---|---|---|
| | IAR in Mineral Oil | Baby Oil |
| Example 6 Corona Treated at | 4.16 | 4.15 |
| 2.5 W/m²/min | (Std Dev. 0.47) | (Std Dev. 0.22) |
| Example 6 Corona Treated at | 4.44 | 4.59 |
| 7.5 W/m²/min | (Std Dev. 0.23) | (Std Dev. 0.18) |

While different types of diaper rash creams may have different ink adhesion ratings, the samples will exhibit improved ink adhesion when corona treated despite the type of diaper rash cream, as shown in Table 7 below. The addition of a reactive lacquer also results in improved ink adhesion.

TABLE 7
Ink Adhesion with Multiple Diaper Rash Creams

| | IAR | IAR | IAR |
|---|---|---|---|
| | HIPOGLOS ® | A + D ® | DESITIN ® |
| | test fluid | test fluid | test fluid |
| Example 6 Corona | 2.30 | 2.17 | 1.63 |
| Treated at | (Std Dev. 0.28) | (Std Dev. 0.19) | (Std Dev. 0.25) |
| 2.5 W/m²/min | | | |
| Example 6 Corona | 2.58 | 2.61 | 1.92 |
| Treated at | (Std Dev. 0.19) | (Std Dev. 0.23) | (Std Dev. 0.37) |
| 7.5 W/m²/min | | | |
| Example 8 Corona | 3.18 | 3.39 | 2.42 |
| Treated at | (Std Dev. 0.18) | (Std Dev. 0.28) | (Std Dev. 0.33) |
| 7.5 W/m²/min and | | | |
| a Reactive Lacquer | | | |
| Example 9 No Corona | 2.20 | 2.27 | 1.60 |
| Treatment, No | (Std Dev. 0.34) | (Std Dev. 0.20) | (Std Dev. 0.25) |
| Reactive Lacquer | | | |

As indicated by the data herein, the ink compositions of the present invention show benefits in the areas of CIE Lab Delta E between the ink printed surface and garment facing surface, ink penetration, leachability, and ink adhesion. Furthermore, even though the ink composition is printed on the inner side of the article layer in some embodiments, the vibrancy is comparable to inks printed in the outer side of the article, while still maintaining high leachability performance.

Additionally, the ink of the present invention does not exhibit bleed through when tested with saline solution or baby oil. Data indicate that the ink compositions of the present invention are better than printed market films or nonwovens. Data further indicate that the ink compositions of the present invention are comparable to printed market films or nonwovens when tested with isopropanol.

In one embodiment of the present invention, once the ink composition has been printed and dried onto the substrate, leachability improves. As shown by the data above, the reactive ink compositions of the present invention are useful in improving leachability. Specifically, the leachability values of the present invention are less than about 3.00×10⁻² absorbance units when tested with baby oil, or less than about 2.00×10⁻³ when tested with saline solution, or less than about 0.14 absorbance units when tested with hexadecane, or less than about 0.10 absorbance units when tested with mineral oil. The ink printed area may have a leachability absorption value of about 0.00 when tested with baby oil or saline solution.

Also, as shown by the data above, the reactive ink compositions of the present invention are useful in improving and balancing the color appearance between the printed and unprinted sides of the nonwoven substrate. Specifically, the Delta E CMC between the printed and unprinted sides of the substrate may be 3.0 or less. In another embodiment, the Delta E CIE may be 5.0 or less.

The color is equally vibrant when viewed from the ink printed surface or the garment facing surface. Printed nonwovens currently in the market appear less vibrant when observed from the garment facing side or non-printed side.

Furthermore, the reactive ink compositions of the present invention are useful in penetrating the fibers of the nonwoven substrate, to achieve a more vibrant color as described by the ink density and color measurements. Imaging analysis performed via light microscopy technique revealed that for the ink of the invention the ink penetrates through the fibers between 35% to 50% of the thickness of the nonwoven. For comparable examples in market, the ink penetrates through the fibers between 3% and 22% of the thickness of the nonwoven.

All documents cited in the Detailed Description of the invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and the scope of the invention. It is therefore intended to cover in the appended claims all such changes and modifications that are within the scope of this invention.

What is claimed is:

1. An absorbent article comprising:
a. a nonwoven substrate having an ink printed inner side and a non-printed garment facing side;
b. a reactive ink composition printed on the nonwoven substrate on the ink printed inner side, forming an ink printed area, wherein the reactive ink composition comprises a binder and an organic solvent.

2. The absorbent article of claim 1, wherein the printed nonwoven substrate is combined with a film and/or a second nonwoven substrate after printing to form a laminate such that the ink printed inner side is in contact with the film and/or the second nonwoven.

3. The absorbent article of claim 1, wherein the reactive ink composition further comprises a crosslinker agent.

4. The absorbent article of claim 3, wherein the crosslinker is selected from the group consisting of poly-isocyanates, blocked poly-isocyanates, poly-epoxy compounds, poly-aziridines, carbodiimides, poly-silanes, and mixtures thereof.

5. The absorbent article of claim 4, wherein the crosslinker is a polyisocyanate.

6. The absorbent article of claim 1, wherein the binder is selected from the group consisting of nitrocellulose, polyamides, polyurethanes, acrylic dispersions, rosin resins, ketone and polyketone resin, polyvinyl butyral, modified celluloses including ethyl cellulose, cellulose esters, cellulose acetate propionate, and cellulose acetate butyrate, polyvinyl alcohol, maleic resin, polyester resin, and mixtures thereof.

7. The absorbent article of claim 1, wherein the binder is a mixture of polyvinyl butyral and nitrocellulose.

8. The absorbent article of claim 1, wherein the ink printed area has a leachability absorption value of less than about 3.00×10⁻² absorbance units when tested with baby oil.

9. The absorbent article of claim 1, wherein the ink printed area has a leachability absorption value of less than about 2.00×10⁻³ when tested with saline solution.

10. The absorbent article of claim 1, wherein the ink printed area has a leachability absorption value of less than about 0.14 absorbance units when tested with hexadecane.

11. The absorbent article of claim 1, wherein the ink printed area has a leachability absorption value of less than about 0.10 absorbance units when tested with mineral oil.

12. The absorbent article of claim 1, wherein the ink printed area of the ink printed inner side has a delta E CMC of less than about 3.0 compared to the non-printed garment facing side.

13. The absorbent article of claim 1, wherein the ink printed area of the ink printed inner side has a delta E CIE of less than about 5.0 compared to the non-printed garment facing side.

14. The absorbent article of claim 1, wherein the ink printed area has an ink penetration from about 25% to about 50% in the fibrous region of a nonwoven.

15. The absorbent article of claim 1, wherein the nonwoven substrate has a basis weight of from about 8 gsm to about 65 gsm.

16. The absorbent article of claim 1, wherein the nonwoven substrate has a basis weight of from about 15 gsm to about 25 gsm.

17. The absorbent article of claim 1, wherein the binder is present in an amount of from about 5% to about 20% of the total solids content.

18. The absorbent article of claim 3, wherein the crosslinker is present in an amount of from about 0.1% to about 10% of the total solids content.

19. The absorbent article of claim 1, wherein the nonwoven substrate is further printed on the garment facing side.

20. The absorbent article of claim 1, wherein the nonwoven substrate is pretreated with a corona treatment.

21. An absorbent article, comprising: a topsheet, a backsheet, and an absorbent core between the topsheet and the backsheet, the backsheet comprising an ink-printed nonwoven substrate, the nonwoven substrate having at least one reactive ink composition printed thereon, forming an ink printed area, wherein the reactive ink composition comprises a crosslinker and a binder, wherein the ink composition is solvent based; wherein the ink printed area has a less than about 3.00×10⁻² absorbance units when tested with baby oil, or less than about 2.00×10⁻³ when tested with saline solution, or less than about 0.14 absorbance units when tested with hexadecane, or less than about 0.10 absorbance units when tested with mineral oil.

22. An absorbent article comprising:
a. a nonwoven substrate having an ink printed garment facing side;
b. a reactive ink composition printed on the nonwoven substrate on the ink printed garment facing side, forming an ink printed area, wherein the reactive ink composition comprises a binder and an organic solvent.

23. The absorbent article of claim 22 wherein the nonwoven substrate is pretreated with a corona treatment.
